(19) United States
(12) Patent Application Publication
(10) Pub. No.: US 2010/0008867 A1
(43) Pub. Date: Jan. 14, 2010

(54) METHOD OF TREATMENT AND PHARMACEUTICAL COMPOSITIONS

(75) Inventors: Howard S. Jaffe, Mill Valley, CA (US); Hans C. Reiser, San Francisco, CA (US); Franck Rousseau, Durham, NC (US); Geoffrey Yuen, Cary, NC (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/487,865

(22) Filed: Jun. 19, 2009

Related U.S. Application Data
(60) Provisional application No. 61/080,139, filed on Jul. 11, 2008.

(51) Int. Cl. A61K 9/12; A61K 31/4725; A61K 31/155; A61K 31/426

(52) U.S. Cl. 424/45; 514/307; 514/635; 514/369

(57) ABSTRACT

The present invention relates to methods, uses, and compositions comprising the caspase inhibitor (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof.

### RELATED APPLICATIONS

This claims priority to U.S. provisional application no. U.S. Ser. No. 61/080,139, filed Jul. 11, 2008.

### FIELD OF THE INVENTION

The present invention relates to methods, uses, and compositions comprising the caspase inhibitor (R)-N-((2S,3S)-2-(fluoromethyl)-2-hydroxy-5-oxo-tetrahydrofuran-3-yl)-5-isopropyl-3-(isoquinolin-1yl)-4,5-dihydroisoxazole-5-carboxamide, or a pharmaceutically acceptable salt thereof.

### BACKGROUND OF THE INVENTION

Fibrosis is the formation or development of excess fibrous connective tissue in an organ or tissue as a reparative or reactive process, as opposed to a formation of fibrous tissue as a normal constituent of an organ or tissue. Fibrosis, much like inflammation, is one of the major, classic pathological processes in medicine. Recognized types of fibrosis include cystic fibrosis of the pancreas and lungs; injection fibrosis, which can occur as a complication of intramuscular injections, especially in children; endomyocardial fibrosis; idiopathic pulmonary fibrosis of the lung; mediastinal fibrosis; myelofibrosis; retroperitoneal fibrosis; progressive massive fibrosis, which is a complication of coal workers' pneumoconiosis; and nephrogenic systemic fibrosis.

Nonalcoholic steatohepatitis, abbreviated as “NASH”, is a liver disease that affects 2 to 5 percent of Americans and resembles alcoholic liver disease, but occurs in people who drink little or no alcohol. NASH is characterized by the presence of fat in the liver, together with inflammation and liver fibrosis. NASH is often asymptomatic, but can progress to cirrhosis, in which the liver is permanently damaged and scarred.

An early indication of NASH is often elevated liver enzymes, such as alanine aminotransferase (ALT) or aspartate aminotransferase (AST). The only means for establishing a diagnosis of NASH is by a liver biopsy. NASH is diagnosed when microscopic examination of the tissue shows the presence of fat along with inflammation and liver damage, such as the formation of fibrotic scar tissue.

NASH can be a silent disease with few or no symptoms. Patients generally feel well in the early stages and only begin to have symptoms—such as fatigue, weight loss, and weakness—once the disease is more advanced or cirrhosis develops. The progression of NASH can take years. The process can stop and, in some cases, reverse on its own without specific therapy, or NASH can slowly worsen, causing scarring to appear and accumulate in the liver. As fibrosis worsens, cirrhosis develops; the liver becomes seriously scarred, hardened, and unable to function normally. A person with cirrhosis may experience fluid retention, muscle wasting, bleeding from the intestines, and liver failure. Liver transplantation is the only treatment for advanced cirrhosis with liver failure. NASH ranks as one of the major causes of cirrhosis in America, behind hepatitis C and alcoholic liver disease

Although NASH has become more common, its underlying cause is still not clear. It most often occurs in persons who are middle-aged and overweight or obese. Many patients with NASH have elevated blood lipids, such as cholesterol and triglycerides, and many have diabetes or pre-diabetes, but not every obese person or every patient with diabetes has NASH. Furthermore, some patients with NASH are not obese, do not have diabetes, and have normal blood cholesterol and lipids. NASH can occur without any apparent risk factor and can even occur in children.

Currently, no approved therapies for NASH exist. Recommendations given to persons with this disease are to reduce their weight (if obese or overweight), follow a balanced and healthy diet, increase physical activity, avoid alcohol and avoid unnecessary medications that may stress the liver.

Accordingly, there is a need for compositions and methods for the treatment and/or prevention of NASH.

### SUMMARY OF THE INVENTION

One aspect of the present invention is a method of treating or preventing NASH, comprising administering to a mammal, such as a human being, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. The compound (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide is a caspase inhibitor that inhibits apoptosis which is a step in the development of NASH. (R)-N-((2S,3S)-2-(fluoromethyl)-2-hydroxy-5-oxo-tetrahydrofuran-3-yl)-5-isopropyl-3-(isoquinolin-1 yl)-4,5-dihydroisoxazole-5-carboxamide accumulates in the liver, and so is concentrated at the site of apoptosis that leads to NASH.

In some embodiments, the mammal (e.g., human being) is suffering from NASH and an amount of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, is administered to the mammal that is effective to treat the NASH. Effective treatment of NASH is typically characterized by the amelioration of at least one symptom of the NASH. For example, an effective amount of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, may slow or stop the progression of fibrosis in the liver of a person suffering from NASH.

### DETAILED DESCRIPTION

The present invention relates to methods, uses, and compositions comprising (R)-N-((2S,3S )-2-(fluoromethyl)-2-hydroxy-5-oxo-tetrahydrofuran-3-yl)-5-isopropyl-3-(isoquinolin-1-yl)-4,5-dihydroisoxazole-5-carboxamide, the structure of which is

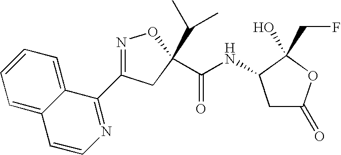

or a pharmaceutically acceptable salt thereof, a caspase inhibitor disclosed in WO 06/90997, herein incorporated by reference.

One aspect of the present invention is a method of treating or preventing NASH, wherein the method comprises the step of administering (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, to a living subject in need thereof (e.g., a human being suffering from NASH).

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by aerosol delivery. In another embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by oral delivery. In another embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered intravenously.

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered in a daily dosage from 1 mg/kg to 500 mg/kg. In one embodiment, a daily dosage is administered from 1 mg/kg to 200 mg/kg. In one embodiment, a daily dosage is administered from 1 mg/kg to 100 mg/kg. In one embodiment, a daily dosage is administered from 1 mg/kg to 80 mg/kg. In one embodiment, a daily dosage is administered in separate sub-doses, namely twice daily or three times daily. In one embodiment, a single daily dosage in the range of from 1 mg/kg to 80 mg/kg is administered once per day.

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof forms part of a combination with an additional therapeutic agent. Examples of other therapeutic agents that can be combined with (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, to treat NASH include insulin sensitizer agents such as thioglitazones (e.g., pioglitazone and rosiglitazone), metformin, glucagon-like peptide-1 (GLP-1) agonists, and antioxidants (e.g., Vitamin E).

Another aspect of the present invention includes the use of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof in the manufacture of a medicament for the treatment or prevention of NASH.

Another aspect of the present invention includes a compound (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, for use in the treatment or prevention of NASH.

Another aspect of the present invention includes a pharmaceutical composition for the treatment of NASH comprising (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof and one or more pharmaceutically acceptable carriers.

In one embodiment, the composition is an aerosol formulation. In one embodiment, the composition is an oral formulation.

In one embodiment, the composition is provided in a daily dosage of from 1 mg/kg to 500 mg/kg, such as from 20 mg/kg to 200 mg/kg, or such as from 10 mg/kg to 100 mg/kg. The daily dosage may be one or more individual doses.

In one embodiment, the composition includes an additional therapeutic agent.

The scope of the present invention includes all combinations of aspects and embodiments.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

In some aspects, the practice of the present invention includes administering (R)-N-((2S,3S )-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, in the pure state or in the form of a composition in which the compound is combined with any other pharmaceutically compatible product, which can be inert or physiologically active. The resulting pharmaceutical compositions can be used to prevent a condition or disorder in a subject susceptible to such a condition or disorder, and/or to treat a subject suffering from the condition or disorder. The pharmaceutical compositions described herein include one or more compounds of Formula 1 and/or pharmaceutically acceptable salts thereof, such as (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof.

The manner in which the compounds are administered can vary. The compositions may be administered orally, namely in liquid form within a solvent such as an aqueous or non-aqueous liquid, or within a solid carrier. Compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Compositions can be in liquid or semisolid form. Compositions including a liquid pharmaceutically inert carrier such as water or other pharmaceutically compatible liquids or semisolids can be used. The use of such liquids and semisolids is well known to those of skill in the art.

The compositions can also be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include 5% dextrose solutions, saline, and phosphate-buffered saline. The compounds can also be administered as an infusion or injection, namely, as a suspension or as an emulsion in a pharmaceutically acceptable liquid or mixture of liquids.

The compounds can also be administered directly to the respiratory tract by inhalation, namely, in the form of an aerosol either nasally or orally. Thus, one aspect of the present invention includes a novel, efficacious, safe, nonirritating, and physiologically compatible inhalable composition comprising (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. As stated herein, such a composition is suitable for treating NASH. Preferred pharmaceutically acceptable salts are inorganic acid salts including hydrochloride, hydrobromide, sulfate or phosphate salts, as they are known to cause less pulmonary irritation. Preferably, the inhalable formulation is delivered to the endobronchial space in an aerosol comprising particles with a mass median aerodynamic diameter (MMAD) between about 1 and about 5 μm. The (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof can be formulated for aerosol delivery using any device capable of producing particles with a mass median aerodynamic diameter (MMAD) between about 1 and about 5 μm. Common examples include nebulizers, pressurized metered dose inhalers (pMDIs), and dry powder inhalers (DPIs).

Non-limiting examples of nebulizers include atomizing, jet, ultrasonic, pressurized, vibrating porous plate or equivalent nebulizers. A jet nebulizer utilizes air pressure to break a liquid into aerosol droplets. An ultrasonic nebulizer works by a piezoelectric crystal that creates standing waves on the surface of the liquid that eject small aerosol droplets. A pressurized nebulization system forces solution under pressure through small pores to generate aerosol droplets via Rayleigh breakup. A vibrating porous plate device utilizes rapid vibration to pump liquid through the porous plate to generate appropriate droplet sizes via Rayleigh breakup.

The amount of active ingredient that may be combined with the excipients to produce a single dosage form that will vary depending upon the host treated and the particular mode of administration. (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is dosed in a therapeutically effective amount ranging from about 1 μg to about 5000 μg. The dose will be determined by the host treated and the severity of the disease as determined by those physicians skilled in the art. Preferably, the drug will be administered four, three, two, or most preferably once a day.

The compounds can also be administered transdermally, such as through use of a transdermal patch or iontophoretically, or by sublingual or buccal administration.

Although it is possible to administer the compounds in the form of a bulk active chemical, it is preferred to present each compound in the form of a pharmaceutical composition or formulation for efficient and effective administration. Exemplary methods for administering such compounds will be apparent to the skilled artisan. The usefulness of these formulations can depend on the particular composition used and the particular subject receiving the treatment. These formulations can contain a liquid carrier that can be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

In an embodiment of the present invention and as will be appreciated by those skilled in the art, the compound of the present invention may be administered in combination with other therapeutic compounds. For example, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof can be used in combination with one or more of the following agents: insulin sensitizer agents such as thioglitazones (e.g., pioglitazone and rosiglitazone), metformin, GLP-1 agonists, and antioxidants (e.g., Vitamin E).

Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof in combination with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The following examples are provided to illustrate the present invention, and should not be construed as limiting thereof. In these examples, all parts and percentages are by weight, unless otherwise noted.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. The precise amount will depend upon numerous factors, for example the particular formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, the specific activity of the composition, the physical characteristics of the composition, its intended use, as well as patient considerations such as severity of the disease state, patient cooperation, etc., and can be determined by one skilled in the art based upon the information provided herein.

Typically, the effective dose of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof generally requires administering the compound in an amount of less than 500 mg/kg of patient weight. The effective doses typically represent that amount administered as a single dose, or as one or more doses administered over a 24-hour period.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993), herein incorporated by reference with regard to such teaching. Where such contextually specific evaluations are relevant, and how they might be relevant in the context of the present invention, will be apparent to one of ordinary skill in the art.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of a disorder mediated through caspase inhibition. The compounds and pharmaceutical compositions of the invention may be used to achieve a beneficial therapeutic or prophylactic effect, for example, in a subject with a need therefor, such as in a human being suffering from NASH.

For example, effective treatment of NASH is typically characterized by the amelioration of at least one symptom of the NASH. For example, an effective amount of (R)-N-((2S,3S )-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, may slow or stop the progression of fibrosis in the liver of a person suffering from NASH. The stage and progression of NASH may be determined using a variety of measurements and assays. For example, the Brunt scoring system may be used to assess the stage of NASH (Brunt E M, et al. Nonalcoholic steatohepatitis: A proposal for grading and staging the histologic lesions. American Journal of Gastroenterology, 94:2467-74 (1999)). Apoptosis in the liver can be measured, for example, using the TUNEL assay (Gavrieli Y, Sherman Y, Ben-Sasson S A., J Cell Biol., 119(3):493-501(Nov. 1992)). Liver inflammation and damage can be assessed, for example, by measuring the amounts of the liver enzymes alanine aminotransferase and aspartate aminotransferase in the blood, wherein elevated levels of one or both of these enzymes indicates inflammation of the liver.

As noted, (R)N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide is a caspase inhibitor disclosed in WO 06/90997, herein incorporated by reference. (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide may be made by a variety of methods. One illustrative synthetic method is set out below. In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3rd Edition, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The compounds can be prepared according to the methods described below using readily available starting materials and reagents. In these reactions, variants may be employed which are themselves known to those of ordinary skill in this art, but are not mentioned in greater detail.

### SYNTHETIC EXAMPLES

As referenced in WO 06/90997, a general preparation for compounds of Formula 1

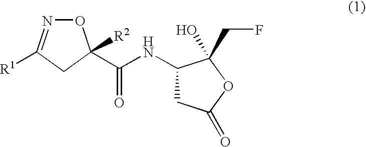

including (R)-N-((2S,3S)-2(fluoromethyl)-2-hydroxy-5-oxo-tetrahydrofuran-3-yl)-5-isopropyl-3-(isoquinolin-1-yl)-4,5-dihydroisoxazole-5-carboxamide provides:

(a) activating a compound of following formula (2),

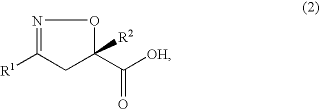

then reacting it with a compound of the following formula (4),

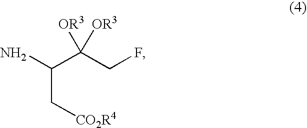

to produce a compound of the following formula (13),

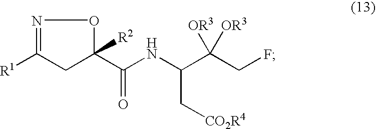

(b) hydrolyzing the compound of the following formula (13) to produce a compound of the following formula (14),

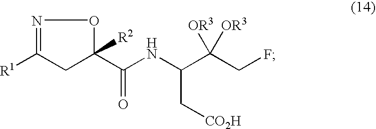

(c) deprotecting the compound of the following formula (14); and (d) carrying out crystallization-induced dynamic transformation. In the above-referenced general process, R¹ is alkyl or aryl; R² is alkyl, each R³ individually is alkyl, or both R³ together with oxygen atom to which they are attached form a heterocycle, and R⁴ is alkyl.

One embodiment provides an activation reagent to activate the compound of formula (2), selected from the group consisting of oxalyl chloride, trimethylacetyl chloride, phosphoryl tri-chloride, and thionyl chloride. Further, step (a) preferably is carried out in the presence of base selected from the group consisting of triethylamine, tri(n-butyl)amine, diisopropy-lethylamine, pyridine, 4-dimethylaminopyridine and 4-(4-methyl-piperidine-l-yl)-pyridine. One preferable ratio provides that the base is used in an amount of 1.0 to 10.0 equivalents to the compound of formula (2). Preferably, the reaction in the step (a) is carried out in one or more solvents selected from the group consisting of dichloromethane, chloroform, tetrahydrofuran, dimethoxyethane, dioxane, and ethyl acetate.

One embodiment provides that the compound of formula (4) in step (a) is used in an amount of 1.0 to 3.0 equivalents to the compound of formula (2). The hydrolysis in step (b) preferably is carried out in the presence of base selected from the group consisting of lithium hydroxide, preferably either anhydrous or monohydrate crystalline, sodium hydroxide, potassium hydroxide, and calcium hydroxide. In one embodiment, the base is used in an amount of 0.1 to 10.0 equivalents to the compound of formula (13).

Preferably, the reaction in the step (b) is carried out in one or more solvents selected from the group consisting of methanol, ethanol, n-propanol, isopropanol, tetrahydrofuran, dimethoxyethane, dioxane, and dichloromethane, or in a mixed solvent including one or more of the solvents selected from the above group and water.

Preferably, the deprotection reaction in the step (c) is carried out in the presence of acid, such as hydrochloric acid, sulfuric acid, or trifluoroacetic acid, and it is preferable that the acid is used in an amount of 0.1 to 20.0 equivalents to the compound of formula (14).

Preferably, the deprotection reaction in the step (c) is carried out in the presence or absence of solvent. If conducted in the presence of solvent, the solvent preferably is selected from dichloromethane or chloroform.

The crystallization-induced dynamic transformation reaction in the step (d) can be carried out by adding the compound of formula (1) as seed, or carried out in the presence of seed and a catalytic amount of base, wherein the base is preferably an amine selected from the group consisting of triethylamine, tri(n-butyl)amine, diisopropylethylamine, diisopropylamine, pyridine, 4-dimethylaminopyridine, 4-(4-methyl-piperidine-l-yl)-pyridine, optically active 1-phenyletheylamine, and optically active 1-naphthylethylamine.

In the step (d), it is preferable to use said amine in an amount of 0.001 to 1.0 equivalent to the compound of formula (14), and more preferable to use 0.03 to 0.5 equivalent. If the amount of used amine is too little, the reaction rate becomes slower, and if the amount is too much, the yield of the compound of formula (1) is decreased.

Further, it is preferable that the crystallization-induced dynamic transformation reaction in the step (d) is carried out in one or more solvents selected from the group consisting of toluene, benzene, dichlorobenzene, tetrahydrofuran, dimethoxyethane, dioxane, ethyl acetate, dichloromethane, acetonitrile, methyl t-butylether, and di-ethylether.

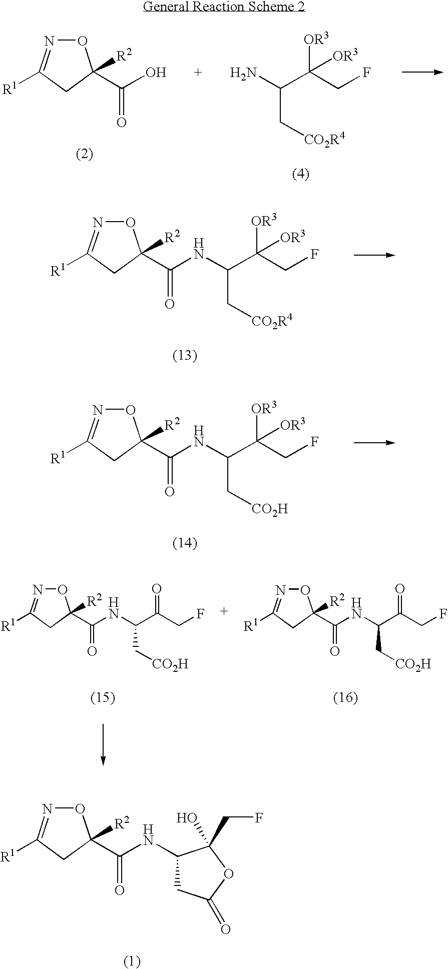

The isoxazoline derivative of formula (2) having high optical activity is prepared according to the process disclosed in PCT/KR2004/02139 filed on Aug. 17, 2004, herein incorporated by reference, and then combined with the compound of formula (4) to produce the compound of formula (13). Then, the compound of formula (13) is ester-hydrolyzed to produce the compound of formula (14), and the deprotection reaction of the ketal moiety of the compound of formula (14) is carried out to obtain a mixture of the compounds of formula (15) and formula (16), which is effectively transformed into the compound of formula (1) by selective dynamic crystallization.

In particular, if the mixture of the compounds of formula (15) and formula (16) is dissolved in organic solvent, and the seed of the compound of formula (1) is added to the solution, only the compound of formula (15) in the mixture is transformed into the compound of formula (1) to be isolated as solid.

Also, if the mixture of the compounds of formula (15) and formula (16) is treated with a catalytic amount of base together with seed, both the compound of formula (15) and the compound of formula (16) are transformed into the compound of formula (1), to produce the compound of formula (1) with higher yield, with reference to General Reaction Scheme 3.

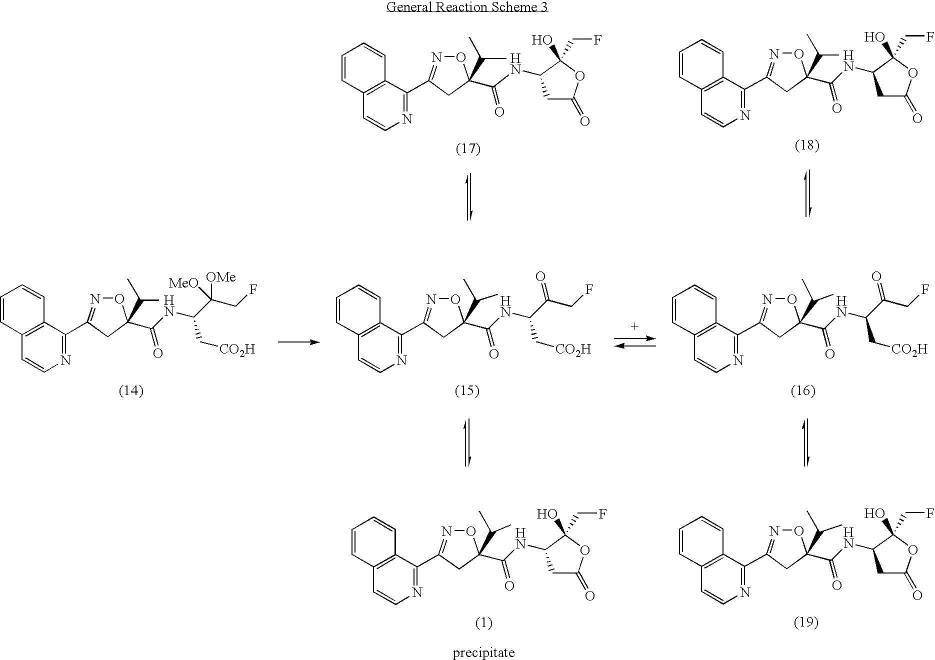

The compound of formula (15) is in equilibrium with the compound of formula (16) due to the base present in solution. Also, the compound of formula (15) is in equilibrium with the compounds of formula (17) and formula (1), and the compound of formula (16) is in equilibrium with the compounds of formula (18) and formula (19). Among them, the compound of formula (1) having good crystallizing property selectively precipitates, and so the equilibrium of all the compounds moves to the compound of formula (1), thereby selectively giving only the compound of formula (1) with high yield from the mixture of the compounds of formula (15) and formula (16).

### Preparation Example 1

### 1-Fluoro-4-trimethylsilanyl-3-butyn-2-one

49.1 g (499 mmol) of trimethylsilyl acetylene was dissolved in 250 mL of anhydrous tetrahydrofuran, and the inner temperature was lowered to about ˜55° C., and then 210 mL (525 mmol) of 2.5 M n-BuLi in n-hexane was added thereto over about 25 minutes with maintaining the inner temperature below −30° C. After stirring for about 40 minutes, 52.9 g (499 mmol) of ethyl fluoroacetate was added to the reaction mixture over 5 minutes with maintaining the inner temperature below −25° C., and then 74.4 g (524 mmol) of BF₃-OEt was added thereto over 15 minutes with maintaining the inner temperature −55° C. to −65° C. After finishing the addition, the reaction mixture was stirred at 20° C. for 2 hours, and 250 mL of 10% ammonium chloride aqueous solution was added thereto to finish the reaction. The organic layer was separated, and the aqueous layer was extracted with 200 mL of ethylacetate. The combined organic phase was washed with 250 mL of brine, and concentrated under reduced pressure. The residue was distilled under vacuum at 10 mbar and 68° C. to give 1-fluoro-4-trimethylsilanyl-3-butyn-2-one

(67.3 g, 85%) as clear oil.

1H NMR (500 MHz, CDCl3): 4.90 (d, J=47.1 Hz, 2H), 0.26 (s, 9H)

¹³C NMR (125 MHz, CDCl3): 181.0 (d, J=21.5 Hz), 104.0, 98.1, 84.8 (d, J=187 Hz)

### Preparation Example 2

### 4-Fluoro-3,3-dimethoxy-1-butyne

33.6 g (316 mmol) of trimethyl orthoformate and 6.0 g (31.5 mmol) of p-TsOH-H₂O together with 50.0 g (316 mmol) of 1-fluoro-4-trimethylsilanyl-3-butyn-2-one obtained from the Preparation Example 1 were put into 260 mL of methanol, and stirred at reflux temperature (inner temperature 60˜64° C.) for about 6 hours. The reaction mixture was concentrated under reduced pressure to remove about 130 mL of solvent, and was diluted with 260 mL of methylene chloride. 130 mL of 10% aqueous sodium hydrogen carbonate solution was added thereto and layer-separated, and the water layer was extracted by using 130 mL of methylene chloride. The combined organic layer was concentrated under reduced pressure to give 4-fluoro-3,3-dimethoxy-l-trimethylsilylbutyne (59.0 g, 92%) as an intermediate, a precursor compound of the object compound 4-fluoro-3,3-dimethoxy-1-butyne. This compound was used in the next reaction without further purification.

¹H NMR (500 MHz, CDCl3): 4.38 (d, J=47.1 Hz, 2H), 3.40 (s, 6H), 0.20 (s, 9H)

59.0 g (289 mmol) of 4-fluoro-3,3-dimethoxy-l-trimethylsilylbutyne, a precursor compound of 4-fluoro-3,3-dimethoxy-1-butyne obtained from the above, was dissolved in 280 mL of methylene chloride, treated with 59 mg (0.183 mmol) of tetra-n butylammoniumbromide and 347 mL (347 mmol) of I N sodium hydroxide aqueous solution, and stirred for about 2 hours. The organic layer was separated, and the aqueous layer was extracted with 110 mL of methylene chloride. The combined organic layer was washed with 110 mL of brine, and concentrated under reduced pressure to give the object compound 4-fluoro-3,3-dimethoxy-1-butyne (40.9 g, quantitative yield). This compound was used in the next reaction without further purification.

¹H NMR (500 MHz, CDCl3): 4.42 (d, J=47.1 Hz, 2H), 3.42 (s, 6H), 2.64 (s, IH)

¹³C NMR (125 MHz, CDCl3): 96.1 (d, J=20.3 Hz), 82.9 (d, J 180 Hz), 77.5, 7.5, 51.0

### Preparation Example 3

### Ethyl 5-fluoro-4,4-dimethoxy-2-pentynoate

A solution of 40.9 g (405 mmol) of diisopropylamine in 270 mL of tetrahydrofuran was cooled to 0° C., and 112 g (405 mmol) of 2.5 M n-BuLi in n-hexane was added thereto over about 1 hour with maintaining the inner temperature below 14° C. The reaction mixture was stirred at 0° C. for about 30 minutes, and the temperature was adjusted to −78° C. A solution of 41.0 g (311 mmol) of the compound 4-fluoro-3,3-dimethoxy-1-butyne obtained from the above Preparation Example 2 dissolved in 160 mL of tetrahydrofuran was added to the reaction mixture over about 2 hours with maintaining the inner temperature below −40° C., and then 60.4 g (557 mmol) of ethyl chloroformate was added thereto over about 1 hour with maintaining the inner temperature below −40° C., and further the reaction mixture was stirred at 0° C. for about 2 hours. 250 mL of 10% ammonium chloride aqueous solution was added to the reaction mixture to finish the reaction, and the organic layer was separated. The aqueous layer was extracted with 100 mL of ethyl acetate, and the combined organic layer was washed with 100 mL of brine and concentrated under reduced pressure to give the crude object compound ethyl 5-fluoro-4,4-dimethoxy-2-pentynoate (95.0 g, calculated yield 70%). This compound was used in the next reaction without further purification

¹H NMR (500 MHz, CDCl3): 4.45 (d, J=46.5 Hz, 2H), 4.25 (q, J=7.1 Hz, 2H), 3.43 (s, 6H), 1.31 (t, J=7.3 Hz, 3H)

### Preparation Example 4

### Ethyl 3-(benzylamino)-5-fluoro-4,4-dimethoxypentanoate

88 g (431 mmol) of the crude compound ethyl 5-fluoro-4,4-dimethoxy-2-pentynoate obtained from the above Preparation Example 3 was dissolved in 430 mL of methyl-t-butyl ether (MTBE), and the temperature was adjusted to 0° C. 31.4 g (293 mmol) of benzylamine was added to the reaction mixture, stirred at 20° C. for about 1 hour, and diluted with 450 mL of methyl-t-butyl ether. Again, the temperature of the reaction mixture was adjusted to 0° C., 33 g (873 mmol) of NaBH₄ was added to the reaction mixture, and then 259 g (4320 mmol) of acetic acid was added thereto over about 30 minutes. The reaction mixture was maintained at 0° C., and 880 mL (2640 mmol) of 3 N sodium hydroxide aqueous solution was slowly added thereto over about 2 hours. The organic layer was separated, and the separated organic layer was washed with 880 mL of 10% ammonium chloride aqueous solution, and then 880 mL of 1 N hydrochloric acid aqueous solution was added thereto. The aqueous layer was separated, washed with 400 mL of methyl-t-butyl ether, and basified by using 246 mL of 10 N sodium hydroxide aqueous solution, and extracted with 700 mL×2 of methyl-t-butyl ether. The combined organic layer was washed with 400 mL of brine, and concentrated under reduced pressure to give the object compound ethyl 3-(benzylamino)-5-fluoro-4,4-dimethoxypentanoate [60.0 g, 44%]. This compound was used in the next reaction without further purification

¹H NMR (400 MHz, CDCl3): 7.35-7.21 (m, 5H), 4.53 (2dd, J=46.8, 10.4 Hz, 2H), 4.13 (q, J=7.2 Hz, 2H), 3.80 (2d, J=12.8 Hz, 2H), 3.53 (dd, J=8.4, 4.0 Hz, IH), 3.30 (s, 3H), 3.22 (s, 3H), 2.79 (dd, J=15.6, 3.6 Hz, IH), 2.40 (ddd, J 15.6, 8.0, 1.6 Hz, IH), 1.25 (t, J=7.2 Hz, 3H)

### Preparation Example 5

### Ethyl 3-amino-5-fluoro-4,4-dimethoxypentanoate

18.3 g (58.5 mmol) of the compound ethyl 3-(benzylamino)-5-fluoro-4,4-dimethoxypentanoate obtained from the above Preparation Example 4 was dissolved in 180 mL of ethanol, and debenzylation was carried out by using activated carbon 5% palladium catalyst (5% Pd/C) at the hydrogen pressure of 50 psi for about 4 hours. The reaction mixture was filtered through 5.0 g of Cellite pad, and washed with 90 mL of ethanol, and the filtrate was concentrated under reduced pressure to give the object compound ethyl 3-amino-5-fluoro-4,4-dimethoxypentanoate (12.8 g, 98%). This compound was used in the next step without any purification.

¹H NMR (500 MHz, CDCl3): 4.53 (2dd, J=46.5, 10.4 Hz, 2H), 4.14 (q, J=7.3 Hz, 2H), 3.57 (dd, J=11.0, 1.9 Hz, IH), 3.29 (d, J=117 Hz, 6H), 2.73 (dd, J=16.5, 2.5 Hz, IH), 2.36 (ddd, J=16.5, 10.4, 2.5 Hz, IH), 1.25 (t, J=7.3 Hz, 3H)

### Preparation Example 6

### 5-fluoro-3-[((R)-5-isopropyl-3-(l-isoquinolinyl)-4,5-dihydro-isoxazole-5-carbonyl)-amino]-4,4-dimethoxy-pentanoic acid ethyl ester

15.5 g (54.5 mmol) of (5R)-5-isopropyl-3-(I-isoquinolinyl)-4,5-dihydro-5-isoxazole carboxylic acid was dissolved in 150 mL of methylene chloride, the temperature was adjusted to 0° C., and then 7.1 mL (81.7 mmol) of oxalyl chloride and 0.2 mL (2.6 mmol) of DMF were added thereto with maintaining the inner temperature below 12° C. The reaction mixture was stirred at 20° C. for about 2 hours, and concentrated under reduced pressure. The reaction mixture was dissolved in 150 mL of methylene chloride, the temperature was adjusted to 0° C., triethylamine was added thereto, and a solution of 12.8 g (57.4 mmol) of the compound ethyl 3-amino-5-fluoro-4,4-dimethoxypentanoate obtained from Preparation Example 5 dissolved in 30 mL of methylene chloride was slowly added thereto over 20 minutes. The reaction mixture was stirred at 25° C. for 1.5 hours, a mixed solution of 120 mL of 10% sodium hydrogen carbonate aqueous solution and 60 mL of 1 N sodium hydroxide aqueous solution was added thereto to finish the reaction. The organic layer was separated, and the aqueous layer was extracted with 150 mL×3 of methylene chloride. The combined organic layer was concentrated under reduced pressure to give the object compound 5-fluoro-3-[((R)-5-isopropyl-3-(l-isoquinolinyl)-4,5-dihydro-isoxazole-5-carbonyl)-amino]-4,4-dimethoxy-pentanoic acid ethyl ester (30.1 g, quantitative yield). This compound was used in the next step without any purification.

¹H NMR (500 MHz, CDCl₃): 9.12 (q, 1H), 8.53 (m, 1H), 7.85-7.25 (m, 4H), 4.80 (m, 1H), 4.54-4.34 (m, 2H), 4.14 (q, J=7.4 Hz, 2H), 3.99 (2d, J=18.4 Hz, 1H), 3.81 (m, 1H), 3.78 (2d, J=8.6 Hz, 1H), 3.33 (d, 3H), 3.20 (d, 3H), 2.75 (m, 3H), 2.53 (m, 1H), 2.39 (heptet, J=6.7 Hz, 1H), 1.27 (t, J=7.4 Hz, 1.5H), 1.07 (m, 6H), 0.97 (t, J=7.4 Hz, 1.5H)

### Preparation Example 7

### 5-Fluoro-3-[((R)-5-isopropyl-3-(l-isoquinolinyl)-4,5-dihydro-isoxazole-5-carbonyl)-amino]-4,4-dimethoxy-pentanoic acid

30.1 g (61.6 mmol) of the compound 5-fluoro-3-[((R)-5-isopropyl-3-(l-isoquinolinyl)-4,5-dihydro-isoxazole-5-carbonyl) -amino]-4,4-dimethoxy-pentanoic acid ethyl ester obtained from the above Preparation Example 6 together with 7.76 g (185 mmol) of lithium hydroxide monohydrate were dissolved in a mixed solvent of 168 mL of tetrahydrofuran and 42 mL of water, and stirred at about 40° C. for 4 hours. The reaction mixture was concentrated under reduced pressure to remove tetrahydrofuran in the solvent, 180 mL of I N sodium hydroxide aqueous solution was added thereto, and the mixture was washed with 120 mL×2 of toluene. The aqueous layer was acidified with 66 mL of 6 N hydrochloric acid aqueous solution, and extracted with 150 mL×3 of methylene chloride, and the combined organic layer was concentrated under reduced pressure to give the object compound 5-fluoro-3-[((R)-5-isopropyl-3-(l-isoquinolinyl)-4,5-dihydro-isoxazole-5-carbonyl) -amino]-4,4-dimethoxy-pentanoic acid (25.4 g, 89%). This compound was used in the next step without any purification.

¹H NMR (400 MHz, CDCl₃): 9.10-8.92 (m, 1H), 8.52 (m, 1H), 7.86-7.13 (m, 4H), 4.77 (m,1 H), 4.54-4.34 (m, 2H), 3.95 (2d, J=8.0 Hz,1H), 3.75 (2d, J=18.4 Hz, 1H), 3.35-3.16 (2d, 6H), 2.78 (2dd, J=16.0, 4.4 Hz 1H), 2.54 (m, 1H), 2.39 (m, 1H), 2.35 (s, 1H), 1.06 (m,6H)

### Example 1

### (45,SS)-5-fluoromethyl-5-hydroxy-4-({[(5 R)-5-isopropyl-3-(l-isoquinolinyl)-4,5-dihydro-5-isoxazolyl]carbonyl}amino)-2-dihydrofuranone

17.0 g (36.9 mmol) of the compound 5-fluoro-3-[((R)-5-isopropyl-3-(l-isoquinolinyl)-4,5-dihydro-isoxazole-5-carbonyl) -amino]-4,4-dimethoxy-pentanoic acid obtained from the above Preparation Example 7 and 6.6 mL (110 mmol) of acetic acid were dissolved in 123 mL (738 mmol) of 6 N hydrochloric acid aqueous solution, and stirred for about 4 hours. The inner temperature of the reaction mixture was adjusted to 0° C., and 150 mL of ethyl acetate was added thereto. 220 mL (660 mmol) of 3 N sodium hydroxide aqueous solution was slowly added to adjust the pH to about 3. The organic layer was separated, and the aqueous layer was extracted with 150 mL×2 of ethyl acetate. The combined organic phase was washed with 100 mL of brine, and concentrated under reduced pressure. The residue was diluted with 50 mL of toluene, and concentrated again under reduced pressure to give a mixture of the compounds of formula (15) and formula (16) as above referenced (15.4 g, quantitative yield, chemical purity: 87.0%).

¹H NMR (500 MHz, DMSO-δ6); 8.99 (m, 1H), 8.65 (m, 1H), 8.19-7.78 (m, 4H), 5.15 (m, 1.5H), 4.77 (m, 1H), 4.42 (m, 0.5H), 3.91 (2d, J=17.6 Hz, 1H), 3.74 (m, 1H), 2.99 (m, 0.2H), 2.82 (m, 1H), 2.63 (m, 0.8H), 2.33 (m, 1H), 0.97 (m, 6H)

To 146 mL of toluene was added 14.6 g (35.2 mmol) of the mixture of the compounds of formula (15) and formula (16) (chemical purity; 87.0%), and the mixture was heated up to 100° C. to dissolve it completely. Then, 14 mg of seed of the object compound was added thereto, the temperature was slowly lowered to 20° C., and the reaction mixture was stirred to produce solid. 0.25 mL (1.8 mmol) of diisopropylamine was added thereto, and stirred at 20° C. for about 2 weeks, to confirm the ratio between the compound of formula (15) and the compound of formula (16)—92.8:7.2 by HPLC. The reaction mixture was concentrated under reduced pressure to remove toluene, 88 mL of ethyl acetate was added thereto, and the mixture was heated up to 65° C. to dissolve it completely. Then, 88 mL of normal hexane was added thereto, and the temperature was slowly lowered and stirred at about 20° C. for 2 days. The resulting solid was filtered, and washed with a mixed solution of 15 mL of ethyl acetate and 15 ml of normal hexane. After drying the solid with nitrogen, the object compound, a white solid was obtained in 54.7% of yield (8.09, chemical purity 98.6%). Solid NMR data of the crystalline form was obtained by using VACP MAS (variable amplitude cross polarization magic angle spinning) at 9 kH spinning rate.

¹H NMR (CDCl₃): 9.02 (bs, 1H), 8.54 (d, J=5.5 Hz, 1H), 7.85 (d, J=7.95 Hz, 1H), 7.70 (m, 3H), 7.60 (bs, 1H), 4.86 (bs, 1H), 4.2-5.2 (bs, 2H), 4.05 (b, J=19.0 Hz, 1H), 3.78 (b, J=19.0 Hz, 1H), 2.7-3.1 (bm, 2H), 2.40 (m, 1H). 1.08 (dd, J=6.7, 4.9 Hz, 6H);

¹³C NMR (CDCl₃): 173.8, 172.4, 160.2, 147.6, 141.7, 136.8, 130.7, 129.0, 127.4, 127.3, 126.8, 122.9, 92.3, 82.7 (d, J=215 Hz), 48.9 (b), 44.6, 34.4, 33.9, 17.7,16.3; ¹³C NMR (solid): 176.4, 171.8, 160.3, 150.2, 139.5, 137.5, 132.3 (2C), 127.7 (3C), 123.0, 104.3, 94.1, 86.4,48.8, 42.9, 32.7 (2C), 19.6, 15.4;

Mass (ESI): 416.14 (M+I).

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method of treating or preventing NASH comprising administering to a human 5 being (R)-N-((2S,3S)-2-(fluoromethyl)-2-hydroxy-5-oxo-tetrahydrofuran-3-yl)-5-isopropyl-3-(isoquinolin-1yl)-4,5-dihydroisoxazole-5-carboxamide or a pharmaceutically acceptable salt thereof.

2. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by oral delivery.

3. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by intravenous delivery.

4. The method of claim 1, further comprising administration of an additional therapeutic agent to the human being.

5. The method of claim 4, wherein the additional therapeutic agent is selected from the group consisting of a thioglitazone, metform in, a GLP-1 agonist, and an antioxidant.

6. The method of claim 1, wherein the (R)-N-((2S,3S)-2-(fluoromethyl)-2-hydroxy-5-oxo-tetrahydrofuran-3-yl)-5-isopropyl-3-(isoquinotin-1-yl)-4,5-dihydroisoxazole-5-carboxamide, or a pharmaceutically acceptable salt thereof, is administered to the human being in a dose of from 1 mg/kg to 500 mg/kg per day.

7. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, is administered to the human being in a dose of from 1 mg/kg to 100 mg/kg per day.

8. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-di hydroisoxazole-5-carboxamide, or a pharmaceutically acceptable salt thereof is administered to the human being once per day.

9. The method of claim 8, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof, is administered to the human being once per day in a dose of from 1 mg/kg to 80 mg/kg.

10. A pharmaceutical composition for the treatment of NASH comprising (R)-N-((2S, 3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof and one or more pharmaceutically acceptable carrier.

11. The pharmaceutical composition of claim 10 wherein the composition is an aerosol formulation.

12. The pharmaceutical composition of claim 10 wherein the composition is an oral formulation.

13. The pharmaceutical composition of claim 10 wherein the composition is an intravenous formulation.

14. The pharmaceutical composition of claim 10 further comprising an additional therapeutic agent.

15. The pharmaceutical composition of claim 14 wherein the additional therapeutic agent is selected from the group consisting of a thioglitazone, metformin, a GLP-1 agonist, and an antioxidant.
